(19) United States
(12) Patent Application Publication
(10) Pub. No.: US 2022/0250095 A1
(43) Pub. Date: Aug. 11, 2022

(54) SEPARATING SYSTEM

(71) Applicant: Cell Therapy Catapult Limited, London (GB)

(72) Inventors: Nicholas Richard GADDUM, London (GB); Mohsen SHAERI, London (GB); Benjamin Andre Frantisek BLAHA, Amsterdam (NL)

(21) Appl. No.: 17/622,567

(22) PCT Filed: Jun. 24, 2020
(86) PCT No.: PCT/GB2020/051529

§ 371 Date: Dec. 23, 2021

(30) Foreign Application Priority Data
Jun. 26, 2019 (GB) 1909210.5

(51) Int. Cl. B04C 11/00; B01D 21/34; B01D 21/26; B01D 21/30; B04C 5/04; B04C 5/12; B04C 5/14; C12N 1/02; C12M 1/26; C12M 1/34

(52) U.S. Cl. CPC B04C 11/00; B01D 21/34; B01D 21/267; B01D 21/302; B04C 5/04; C12M 41/40; B04C 5/14; C12N 1/02; C12M 33/10; C12M 41/36; B04C 5/12

(57) ABSTRACT

A separating system, for example for separating material from a suspension such as a biological suspension, is disclosed herein. The system comprises a separation vessel arranged to enable the formation of a cyclone therewithin. For example, the separation vessel may be at least partially conical in shape for enabling the formation of a cyclone therewithin. The separation vessel comprises a fluid inlet, an underflow outlet and an overflow outlet. The system also comprises at least one of an underflow outlet fluid control means for controlling the flow of fluid through the underflow outlet, and an overflow outlet fluid control means for controlling the flow of fluid through the overflow outlet. The system may further comprise an inlet fluid control means for controlling the flow of fluid through the fluid inlet.

### FIELD OF THE INVENTION

The present disclosure relates to a separating system, method and controller, such as a system, method and controller for separating material from a suspension, such as cells or beads from a biological suspension.

### BACKGROUND

The expansion of cells may be desirable for many applications, including the manufacture of cell therapies, for example in both allogeneic and autologous cell therapies, to allow the generation of product to meet patient demand. Media replacement is a requirement for many cell culture systems, to allow optimal product development and yield, and the use of efficient cell culture perfusion systems, which allow the maintenance of a specific environment in a culture vessel or bioreactor, is important for the production of cell therapies. Additionally, there exists a requirement to concentrate cells after expansion into an appropriate volume for downstream applications to enable product formulation. Particularly, cell therapies often require the culture and expansion of cells from a single individual, for ultimate administration to multiple patients, necessitating intensified cell expansion (perfusion) and an ultimate formulation (volume reduction).

Hydrocyclones are devices which are commonly used to separate or sort particles from a fluid suspension, typically involving the formation of a cyclone to achieve separation of larger and/or denser material from the fluid in which it is suspended. The devices are very simple with no moving parts, are robust and sterilisable in situ. However, their use in real life can be problematic due to the different conditions and environments in which they are used affecting the formation of a cyclone inside the device, and therefore the efficacy of such devices, requiring careful balancing and fine tuning every time a change is made to the system. The use of hydrocyclones is particularly problematic when scaling up where the use of large volumes of suspension can result in a low yield or recovery of product.

### SUMMARY OF THE INVENTION

Aspects of the invention are as set out in the independent claims and optional features are set out in the dependent claims. Aspects of the invention may be provided in conjunction with each other and features of one aspect may be applied to other aspects.

A typical hydrocyclone device in use comprises a pump pumping in a fluid through an inlet into a separation vessel. The separation vessel may be elongate and symmetrical (for example, the separation vessel may have a conical or tubular shape) and therefore have a longitudinal axis extending in the elongate direction. The fluid is fed into the separation vessel transverse to (for example, perpendicular to) and eccentric to a longitudinal axis of the separation vessel such that it creates a cyclone effect in the vessel centred about (and therefore coaxial to) the longitudinal axis. Denser and/or more massive particles in the fluid travel around the sides of the separation vessel and out through an outlet at one end of the separation vessel (the underflow outlet), and fluid comprising fewer particles, or less dense particles with a smaller mass, are forced through an outlet at another end of the separation vessel (the overflow outlet). The ratio of the diameters of the underflow outlet, Du, to the overflow outlet, Do, has been discussed as critical in the literature. These determine the relative resistance to flow (Ru/Ro) through the cyclone. In addition, the inlet pressure is also an important determinant of cyclone function driven by the pump operation, and Di the inlet diameter. These drive the flow rates which are Qu (flow rate from the underflow) to Qo (flow rate from the overflow), and which in turn have an effect on the concentration (or yield).

However, when the hydrocyclone is attached to other equipment via tubing etc., the ratio of Du to Do is not the only variable generating the resistance to flow-tube lengths, connectors/valves, tortuous fluid paths (corners/twists), and gravity effects with return and waste reservoirs at different heights etc, can all have an effect.

From a commercial standpoint, controlling the resistance that an end-user applies to a supplied hydrocyclone is very difficult, even if complete kits are provided—and could easily change the resistance balance rendering the hydrocyclone ineffective. This problem has not yet been recognised in the art.

Embodiments of the disclosure involve an adaptation of the conventional hydrocyclone separation system to include at least one flow control means, such as a resistance control (such as a valve) and/or pressure/flow control (such as a pump e.g. a syringe pump), at the overflow and/or underflow outlets of the separation vessel. This allows control of the system meaning that the system does not have to be adapted to consider Ru or Ro (or even Du or Do). The separation device can be used for any type of system, regardless of tubing or size and does not need to be carefully balanced and fine-tuned to take into account Ru or Ro, unlike the systems of the prior art. Thus, the separation system developed by the inventors is extremely advantageous over the prior art systems, which at worst would not work upon scale up, or at best require fine tuning and balancing every time a change is made to the system.

Although, hydrocyclones have been used in other large-scale industries without controlling Ru to Ro, such industries are generally those where yield is not critical. In industries where yield is extremely important, for example the cell therapy industry, the use of an optimally tuned separation system, such as a hydrocyclone, is critical and thus the presently developed separation system represents a system which could be employed within such yield-critical industries. However, it is noted that the device could also have utility in other industries, particularly those where an increased yield would be desirable, if not critical.

It is also noted that while the formation of a cyclone (i.e. the dynamic rotating fluid structure) in the separation system may be preferable, this is not always necessary to achieve separation of denser particles/material from a fluid suspension.

Accordingly, in a first aspect there is provided a separating system, for separating material (e.g. cells and/or beads) from a suspension, particularly from a biological suspension, such as a cell suspension. The system comprises a separation vessel arranged to enable the formation of a cyclone therewithin. For example, the separation vessel may be at least partially conical in shape for enabling the formation of a cyclone therewithin. However, it will be understood that the formation of a conical cyclone in the separation vessel may not be essential for enabling separation to occur.

The separation vessel comprises a fluid inlet, an underflow outlet and an overflow outlet.

The system also comprises at least one of (and in some examples, both of) an underflow outlet fluid control means for controlling the flow of fluid through the underflow outlet, and an overflow outlet fluid control means for controlling the flow of fluid through the overflow outlet.

It will be understood that when reference is made to the terms underflow outlet and overflow outlet, these are described with reference to the separation vessel and in particular, in use, to the longitudinal axis of a cyclone formed in the separation vessel and therefore with reference to where fluid/material is extracted from the separation vessel along the longitudinal axis of the cyclone. As such, the underflow outlet may be described as an outlet that is configured to draw fluid from one point along the longitudinal axis (such as proximal to one end of the longitudinal axis), and the overflow outlet may be described as an outlet that is configured to draw fluid from a second point along the longitudinal axis (such as proximal to the other end of the longitudinal axis, and distal to the underflow outlet). It will be understood that typically the fluid inlet is located somewhere along the longitudinal axis of the cyclone, typically in use proximal to the overflow outlet at a distal end of the longitudinal axis of the cyclone. It will also be understood that the longitudinal axis of the cyclone typically corresponds to (for example, is parallel to and coaxial with) the longitudinal axis of the separation vessel, which is also an axis of symmetry of the separation vessel, particularly in examples where the separation vessel is conical or cylindrical. It will also be understood that one of the outlets may have a larger diameter (for example to accommodate a greater flow rate) than the other.

In some examples it will be understood that the separation vessel may comprise a plurality of fluid inlets and/or a plurality of underflow outlets and/or a plurality of overflow outlets. In examples where there are a plurality of fluid inlets and/or a plurality of underflow outlets and/or a plurality of overflow outlets, at least one of the fluid inlet/outlets may be larger than the other, for example having an aperture with a larger cross-sectional area. For example, the separation vessel may comprise a single overflow outlet (for example for waste), and two concentric underflow outlets (for example for taking different cell mass fractions). For example, larger cells/clusters may be extracted through a wider underflow outlet, and smaller/single cells through a narrower underflow outlet.

The system may further comprise an inlet fluid control means for controlling the flow of fluid through the fluid inlet. In some examples therefore the system comprises at least two of, and optionally all three of, an inlet fluid control means for controlling the flow of fluid through the fluid inlet, an underflow outlet fluid control means for controlling the flow of fluid through the underflow outlet, and an overflow outlet fluid control means for controlling the flow of fluid through the overflow outlet.

It will be understood that the inlet fluid control means, the underflow outlet fluid control means and/or the overflow outlet fluid control means may be configured to adjustably vary the flow rate and/or pressure of fluid flowing through the corresponding inlet or outlet, for example based on a predefined relationship between the flow rate through the fluid inlet, the underflow outlet and/or the overflow outlet. For example, the fluid control means may be operable to proportionately increase or decrease the flow rate of fluid through one of the overflow outlet and the underflow outlet based on a proportionate change in flow rate through the fluid inlet—in short, the fluid control means may be operable to variably control the fluid flow rate and/or pressure.

The system may further comprise a controller for controlling at least one of:
(i) the underflow outlet fluid control means;
(ii) the overflow outlet fluid control means.

In some examples the system comprises a controller for controlling at least one of:
(iii) the underflow outlet fluid control means;
(iv) the overflow outlet fluid control means; and
(v) the inlet fluid control means.

Typically a single controller may control all of the fluid control means present, however it will be understood that in other examples a separate controller may be provided for controlling each respective fluid control means.

The controller may be configured to control at least one of: (i) the underflow outlet fluid control means, and (ii) the overflow outlet fluid control means based on the flow rate and/or pressure of fluid through the fluid inlet to control the separation of material from the biological suspension. In some examples the controller may be configured to control at least one of: (i) the underflow outlet fluid control means, and (ii) the overflow outlet fluid control means such that the flow rate of fluid through the underflow outlet is greater than the flow rate of fluid through the overflow outlet.

In some examples the system further comprises at least one sensor coupled to the controller and arranged to sense a parameter of the fluid flowing through at least one of the fluid inlet, the underflow outlet and the overflow outlet. In some examples there may also be a sensor inside one of the vessels, such as the separation vessel, the feed vessel and/or the waste vessel. The controller may be configured to make a determination of a parameter of the fluid based on sensor signals received from the at least one sensor, and control at least one of the fluid control means based on the determination/sensor signals received from the at least one sensor.

In some examples the controller may comprise a closed loop control system. For example, the controller may comprise a feedback control loop to control at least one of the fluid control means based on sensor signals received from a sensor as a continuous process. For example, the controller may control at least one of the fluid control means based on sensor signals received from a sensor to maintain the fluid at a point in the system at a particular state (for example to maintain a particular density, impedance, conductance and/or pressure). However, it will be understood that in other examples the controller may be part of an open loop control system.

For example, the system may comprise a first sensor coupled to the controller and arranged to sense a parameter of the fluid flowing through the fluid inlet; and a second (or more) sensor coupled to the controller and arranged to sense a parameter of the fluid flowing through at least one of: (i) the underflow outlet, (ii) the overflow outlet and (iii) inside the separation vessel. In such examples the controller may be configured to control the inlet fluid control means and at least one of: (iv) the underflow outlet fluid control means, and (v) the overflow outlet control means, based on sensor signals received from the sensors.

For example, the controller may be configured to keep the flow rate of fluid through at least one of the outlets above a selected threshold, and if the controller determines that the flow rate falls below the selected threshold, the controller may be operable to control a fluid control means to increase the flow rate, Similarly, the controller may additionally or alternatively be configured to control the separation process based on the density and/or size of particles flowing through the fluid through at least one of the fluid inlet, the overflow outlet and/or the underflow outlet. For example, if the controller determines that the density and/or size reaches and/or exceeds a selected threshold, the controller may be operable to control a fluid control means to increase the flow rate through at least one of the underflow and overflow outlets.

The sensors may be selected from at least one of: a turbidity sensor, a temperature sensor, a pressure sensor, a flow sensor, a capacitive sensor and an impedance sensor. The flow sensor may comprise a magnetic flow meter (for example that measurement distortions in an induced magnetic field due to flow), a Coriolis flow meter, an ultrasonic flow meter (for example Doppler flow meter or a time of flight flow meter), and/or a mechanical flow meter (for example using a paddle wheel, turbine or variable flow area). In some examples the sensor may comprise a camera or other similar means for capturing images of the fluid flow, and the camera may be coupled to a processing means such as a computer for processing the images to determine properties such as flow rate. In some examples the sensor may be a capacitive sensor. For example, the sensor may be configured to perform capacitive biomass measurement to determine the viable cell biomass in the fluid by way of generating an electric field in the cell suspension. This causes the cells to polarise, with the more cells being present, the greater the degree of polarisation, which in turn affects the observed capacitance measurement.

In some examples, at least one of the sensors is a turbidity sensor, and the controller may be configured to make a determination of the density of the fluid based on sensor signals received from the turbidity sensor, and control at least one of the fluid control means based on the determined density of the fluid.

In some examples the system further comprises a feed vessel, for example for containing a suspension such as a biological suspension, coupled to the fluid inlet. In use the feed vessel may comprise a suspension, particularly a biological suspension, comprising material such as cells, cell media and/or beads. The controller may be configured to control the pressure of the feed vessel for controlling the flow of fluid through the fluid inlet. For example, the controller may be configured to control a compressed gas feed to the feed vessel to control the pressure in the feed vessel. For example, the controller may be configured to keep the pressure in the feed vessel above a selected threshold, and if the controller determines that the pressure in the feed vessel drops below the selected threshold, the controller may be operable to control the compressed gas feed to supply more compressed gas to the feed vessel to increase the pressure in the feed vessel.

In addition, and as will be described in more detail below, in some examples at least one of the underflow and/or overflow outlets may be coupled to respective harvest and waste vessels. The controller may be configured to control the pressure of the harvest and/or waste vessels for controlling the flow of fluid through the respective underflow/overflow outlets. For example, the controller may be configured to keep the pressure in the harvest and/or waste vessels below a selected threshold, and if the controller determines that the pressure in the waste and/or harvest vessels reaches and/or exceeds the selected threshold, the controller may be operable to control a fluid control means to reduce the pressure in the harvest and/or feed vessel.

The fluid control means may comprise energy addition means, such as a pump (for example to control the pressure in a feed, waste or harvest vessel, or directly pumping fluid into or out of the separation vessel). The fluid control means may also comprise energy reduction means, such as a fluid resistor, for example an actuated pinch valve. The fluid control means may also comprise passive means, such as flexible tubes which respond to increased pressure by expanding.

For example, at east one of the fluid control means may comprise a continuous pump such as a rotary pump, a non-continuous pump such as a syringe pump, and/or a fluid resistor (which may comprise flexible tubing that can be adjusted to alter the degree of resistance the tubing provides to the flow of fluid therethrough).

It will also be understood that at least one of the fluid control means may comprise any other means, mechanical or otherwise, capable of controlling the flow of fluid. For example, a fluid control means may comprise a means for controlling the pressure in the system (such as in the feed vessel, harvest vessel or waste vessel as described in more detail below) for controlling the flow of fluid in the system. In such examples the controller may be configured to control the flow rate of fluid by controlling a pressure (either positively or negatively) of the fluid being fed into and/or extracted from the separation vessel. This may comprise, for example, controlling the pressure in the feed vessel, the harvest vessel and/or the waste vessel.

In another aspect there is also provided a separating method, for example a method for separating material, such as cells and/or beads such as alginate beads, from a suspension, particularly from a biological suspension, such as a cell suspension. The method comprises feeding a fluid, for example a suspension (e.g. a biological suspension) containing material such as cells, into a separation vessel via a fluid inlet for establishing a cyclone in the vessel, wherein the separation vessel comprises an underflow outlet and an overflow outlet. The fluid is fed into the separation vessel transverse to (for example, perpendicular to) and eccentric to a longitudinal axis of the separation vessel such that it creates a cyclone effect in the vessel to form a cyclone about the longitudinal axis of the separation vessel.

The method also comprises controlling the flow of fluid through at least one of the underflow outlet and the overflow outlet to control the separation of material from the suspension. In some examples the flow of fluid through the fluid inlet and at least one of the underflow outlet and the overflow outlet is controlled.

Controlling the flow of fluid may comprise controlling the flow rate of fluid. In some examples the method comprises controlling the pressure and/or speed of the fluid, such as the suspension, fed into the vessel to control the formation of a cyclone in the separation vessel and/or the extraction/separation of material from the fluid.

In some examples controlling the flow of fluid may comprise controlling the flow rate and/or pressure of fluid fed into the separation vessel via the fluid inlet to be above a selected inlet threshold flow rate and/or pressure. For example the pressure of the feed vessel can be adjusted to control the flow of fluid into and/or out of the separation vessel and thereby the flow rate and/or pressure, for example to provide a static head pressure to the fluid inlet that is above an inlet threshold pressure. The inlet threshold flow rate and/or pressure may be selected based on at least one of (i) the dimensions of the separation vessel, (ii) the relative positions of the fluid inlet, underflow outlet and overflow outlet and/or the minimum and (iii) the minimum or maximum size of cyclone that can be formed and supported inside the separation vessel. In such examples, the flow of fluid through at least one of the underflow outlet and the overflow outlet may be controlled to adjust the rate of separation performed (in other words the amount of material extracted from the fluid as a function of the fluid flow rate) by the separation vessel, and therefore the separation efficiency of the system.

In some examples, the flow rate and/or pressure of fluid via the fluid inlet may be controlled based on the flow rate of fluid through at least one of, and optionally through both of the, underflow outlet and the overflow outlet, or vice-versa, so that the flow rate and/or pressure of fluid through at least one of, and optionally through both of the, underflow outlet and the overflow outlet, may be controlled based on the flow rate and/or pressure of fluid via the fluid inlet. However it will be appreciated that it is not possible to control the flow rate through all of the fluid inlet, the underflow outlet and the overflow outlet simultaneously due to the conservation of mass of an incompressible fluid.

For example, the flow rate and/or pressure of fluid via the fluid inlet may be controlled as a function (for example, proportionate to) the flow rate and/or pressure of fluid through at least one of, and optionally through both of the, underflow outlet and the overflow outlet. The relationship between the flow rate and/or pressure of fluid via the fluid inlet to the flow rate and/or pressure of fluid through at least one of, and optionally through both of the, underflow outlet and the overflow outlet, may be held in a look up table accessed by a controller controlling the flow rate and/or pressure of fluid through the fluid inlet, the underflow outlet and/or the overflow outlet.

In some examples preferably the flow of fluid through the overflow outlet is controlled. Controlling the flow of fluid through the overflow outlet may help to control the formation of a cyclone in the separation vessel, although it will be understood that controlling the formation of a cyclone in the separation vessel may additionally or alternatively be performed by controlling the flow of fluid through the fluid inlet.

Controlling the flow of fluid through the overflow outlet may also control the degree of separation of material from the fluid, and where within the separation vessel (and therefore, in use, from within a cyclone formed in the separation vessel) fluid is extracted from. For example, the size of the area surrounding the overflow outlet from which fluid is extracted may be dependent on the flow rate of fluid through the overflow outlet. If fluid is extracted from the overflow outlet more quickly, the area from (in use, from within the cyclone) which the fluid is extracted increases, and fluid may therefore be extracted from nearer the periphery of the cyclone and therefore contain suspended material/particles with a greater mass/that are more dense as opposed to material/particles extracted nearer the centre of the cyclone (with respect to the longitudinal axis of the cyclone). By contrast, if fluid is extracted from the overflow outlet more slowly, then the area from which the fluid is extracted (in use, from within the cyclone) decreases, and fluid may therefore be extracted from nearer the centre of the cyclone (with respect to the longitudinal axis of the cyclone) and therefore contain suspended material/particles with a lower mass/that are less dense, as opposed to material/particles extracted nearer the periphery of the cyclone (with respect to the longitudinal axis of the cyclone).

The method may further comprise receiving sensor signals indicative of a parameter of the fluid flowing through at least one of: (i) the fluid inlet, (ii) the underflow outlet, (iii) the overflow outlet, and (iv) inside the separation vessel. The method may comprise controlling the flow of fluid through at least one of: (v) the underflow outlet, and (vi) the overflow outlet, to control the degree and/or rate of separation of material from the suspension in the separation vessel based on the received sensor signals. The method may additionally or alternatively comprise controlling the flow of fluid into the separation vessel via the fluid inlet, for example based on the received sensor signals.

The method may comprise controlling the flow of fluid through at least one of: (i) the underflow outlet, and (ii) the overflow outlet, based on the flow rate and/or pressure of fluid through the fluid inlet to control the separation of material from the biological suspension. For example, the method may comprise varying the flow rate of fluid through at least one of the underflow outlet and the overflow outlet based on a change in flow rate through the fluid inlet. In some examples the method may comprise controlling at least one of the fluid control means, and typically at least two of (such as the fluid inlet control means and the overflow outlet fluid control means) to achieve a desired target degree and/or rate of separation of material from the biological suspension. For example, the flow rate of fluid through the fluid inlet may be controlled by the fluid inlet control means, and the flow rate of fluid through the overflow outlet may be controlled by the overflow outlet fluid control means, based on a predefined relationship between the two flow rates (wherein the relationship may also be based on the dimensions of the separation vessel) to reach a desired degree and/or rate of separation of material from the suspension. For example, the predefined relationship may be held in a lookup table. The relationship may be linear (e.g. proportional) or non-linear, and may be a mathematical relationship.

In another aspect there is provided a controller for controlling a separating system, for example for controlling the separation of material from a suspension, e.g. for controlling the separation of cells and/or beads from a biological suspension. The separating system may comprise a separation vessel having a fluid inlet, an overflow outlet and an underflow outlet, and the controller is configured to control the flow rate of fluid through at least one of: (i) the overflow outlet, and (ii) the underflow outlet, based on the flow rate and/or pressure of fluid through the fluid inlet to control the separation of material from the biological suspension. For example, the controller may be configured to reference a lookup table that stores the relationships between the different values of the flow rates of the fluid inlet, overflow outlet and/or underflow outlet and the degree and/or rate of separation of material from the suspension achieved for the differing values of the flow rates at each of the fluid inlet, the underflow outlet and/or the overflow outlet. For example the controller may be configured to alter the flow rate and/or pressure through one of the underflow outlet and overflow outlet based on a change in flow rate and/or pressure through the fluid inlet. For example, the controller may be configured to control the flow rate of fluid through the fluid inlet by operating the fluid inlet control means, and the flow rate of fluid through the overflow outlet by operating the overflow outlet fluid control means, based on a predefined relationship between the two flow rates (wherein the relationship may also be based on the dimensions of the separation vessel) to reach a desired degree and/or rate of separation of material from the suspension. For example, the predefined relationship may be held in a lookup table referenced by the controller.

In another aspect there is provided a controller for controlling a separating system, for example for controlling the separation of material from a suspension, for example for controlling the separation of cells and/or beads from biological suspensions. The separating system may comprise a separation vessel having a fluid inlet, an overflow outlet and an underflow outlet, and the controller is configured to control receive sensor signals indicative of a parameter of the fluid flowing through at least one of the fluid inlet, the underflow outlet and the overflow outlet. The controller is configured to control, based on sensor signals received from the sensor, at least one of: (i) an underflow outlet fluid control means for controlling the flow of fluid through the underflow outlet, and (ii) an overflow outlet fluid control means for controlling the flow of fluid through the overflow outlet.

For example, the controller may be configured to control the flow rate and/or pressure of fluid flowing through at least one (and optionally both of) the underflow outlet and the overflow outlet based on the flow rate and/or pressure of fluid flowing through the fluid inlet. The controller may be configured to do this to control the separation of material from the biological suspension. For example, the controller may be configured to reference a lookup table that stores the relationships between the different values of the flow rates of the fluid inlet, overflow outlet and/or underflow outlet and the degree and/or rate of separation of material from the suspension achieved for the differing values of the flow rates at each of the fluid inlet, the underflow outlet and/or the overflow outlet. For example the controller may be configured to alter the flow rate and/or pressure through one of the underflow outlet and overflow outlet based on a change in flow rate and/or pressure through the fluid inlet. For example, the controller may be configured to control the flow rate of fluid through the fluid inlet by operating the fluid inlet control means, and the flow rate of fluid through the overflow outlet by operating the overflow outlet fluid control means, based on a predefined relationship between the two flow rates (wherein the relationship may also be based on the dimensions of the separation vessel) to reach a desired degree and/or rate of separation of material from the suspension. For example, the predefined relationship may be held in a lookup table referenced by the controller. The relationship may be a linear one or may be non-linear. The relationship may be a mathematical one.

The controller may further be configured to control an inlet fluid control means for controlling the flow of fluid through the fluid inlet based on the received sensor signals.

In some examples the controller is configured to determine the particle density within the fluid (such as the cell density), and optionally control the flow of fluid based on the determined particle density of the fluid. For example, the controller may be configured to control the flow rate of fluid through at least one of the fluid control means based on the determined particle density of the fluid. Additionally or alternatively, the controller may be configured to control the flow rate of fluid by controlling a pressure of the fluid being fed into the separation vessel, for example by controlling a pressure of a feed vessel. It will also be understood that the controller may be configured to determine the size and/or mass of particles within the fluid, and optionally control the flow of fluid as described above based on the determined size and/or mass of the particles.

In some examples where the density, size and/or mass of particles in the fluid are indirectly sensed, for example using indirect flow/pressure sensors (as opposed to direct particle density measurement using capacitance, turbidity etc.), the controller may be configured to control the fluid control means according to a priori knowledge that a cyclone with a specific geometry, and running at a specific inlet flow/pressure would require a specific flow rate of fluid through, for example the overflow outlet and/or underflow outlet. The controller may also be configured to determine an optimal inlet flow rate vs overflow and/or underflow outlet flow rate. This could be expressed in terms of a fitted function, or for example contained in a lookup table that may be referenced by the controller, upon which the overflow rate could be adjusted accordingly.

The controller may be configured to operate in two modes:
(i) an initialisation mode for establishing a cyclone in the separation vessel; and
(ii) a cyclone mode for separating material from a suspension, such as cells from a cell suspension.

In the initialisation mode the controller may be configured to inhibit the flow of fluid through the overflow outlet, such that no fluid flows through the overflow outlet (for example such that the overflow outlet is blocked); and in the cyclone mode the controller may be configured to adjustably control the flow of fluid through at least one of (i) the overflow outlet and (ii) the underflow outlet. The controller may be configured to determine when to switch between the initialisation mode and the cyclone mode based on a parameter, such as the speed and/or pressure, of the fluid passing through at least one of (i) the underflow outlet and (ii) the overflow outlet. Preferably the controller is configured to switch between the initialisation mode and the cyclone mode based on a parameter of the fluid passing through the overflow outlet. When the controller switches between the initialisation mode and the cyclone mode, the controller may be configured to gradually increase or ramp up the flow rate of the flow of fluid through the overflow outlet to a selected rate, for example from a flow rate of zero (i.e. blocked) to the selected flow rate. This may be desirable so as not to create any sudden/destabilising perturbations to the system which may result in the cyclone collapsing.

In another aspect there is provided a computer program comprising instructions to cause the controller of any of the aspects described above to perform the method of the aspect described above.

The position of the fluid inlet, the overflow outlet and/or the underflow outlet may be selected based on the dimensions of the separation vessel and the size/dimensions of a cyclone that can be formed inside, and supported by, the separation vessel. For example, the position of the overflow outlet may be selected to be distal of the fluid inlet and the underflow outlet along the longitudinal axis of the separation vessel (where the underflow outlet may be considered to be proximal along the longitudinal axis of the separation vessel). However, in other examples the overflow outlet (and in particular, the point from which inside the separation vessel the overflow outlet draws fluid) may be positioned between the underflow outlet and the fluid inlet along the longitudinal axis of the separation vessel. The exact position of the overflow outlet, for example, along the longitudinal axis may be selected based on the desired degree and rate of separation.

### DRAWINGS

Embodiments of the disclosure will now be described, by way of example only, with reference to the accompanying drawings, in which:

FIG. 1 shows a functional schematic view of an example separation system;

FIG. 2 shows another functional schematic view of an example separation system;

FIG. 3 shows another functional schematic view of an example separation system;

FIG. 4 shows another functional schematic view of an example separation system:

FIG. 5 shows another functional schematic view of an example separation system;

FIG. 6 shows another functional schematic view of an example separation system; and

FIG. 7 shows another functional schematic view of an example separation system.

### SPECIFIC DESCRIPTION

Embodiments of the disclosure relate to a separating system, for example for separating material such as cells and/or beads from a suspension such as a cell suspension, although it will be understood that the separating system may find application in other fields of use. The system involves an adaptation of a conventional hydrocyclone system to include a flow control means that allows control of the system meaning that the system does not have to be adapted to consider Ru or Ro (or even Du or Do). The separation system can be used for any type of system, regardless of tubing or size and does not need to be carefully balanced and fine-tuned to take into account Ru or Ro, unlike the systems of the prior art.

In the cell therapy industry where yield is extremely important, the use of an optimally tuned separation device is critical and thus the presently developed hydrocyclone will be important to the industry. However, it is noted that the device could also have utility in other industries, particularly those where an increased yield would be desirable.

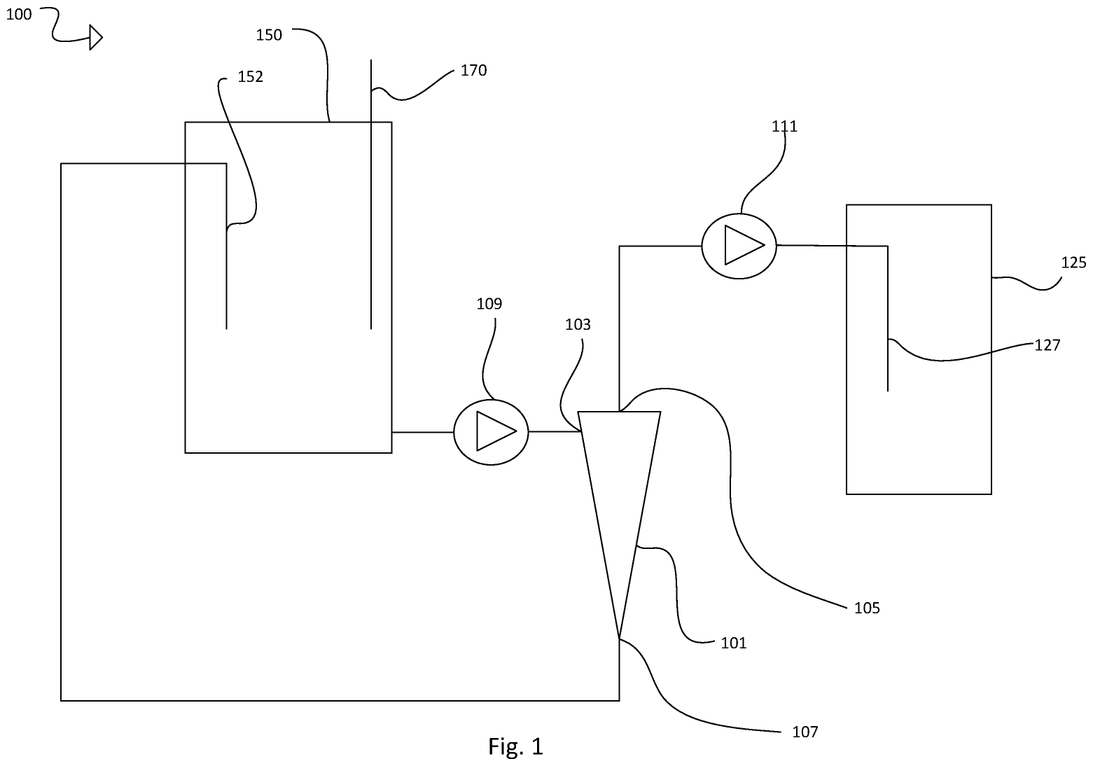

FIG. 1 shows an example separating system 100 of embodiments of the disclosure. The system 100 comprises a separation vessel 101 having a fluid inlet 103, an underflow outlet 107 and an overflow outlet 105. In the example shown the separation vessel 101 is conical in shape to enable the formation of a cyclone therewithin, however it will be understood that in other examples the separation vessel 101 may have another shape. The fluid inlet 103 is proximate to the overflow outlet 105 and configured to direct fluid into the vessel transverse to (for example, perpendicular to) and eccentric to a longitudinal axis of the conical separation vessel 101 such that it creates a cyclone effect in the vessel about the longitudinal axis. The fluid inlet may have a diameter between 1.0 and 4.0 mm. The underflow outlet 107 and overflow outlet 105 are coaxial with the longitudinal axis of the conical separation vessel 101. The underflow outlet 107 may have a diameter between 0.1 and 3.0 mm, preferably 0.1 to 1.0 mm, and the overflow outlet 105 may have a diameter in the range of 0.1 to 3.0 mm, or preferably 0.1 to 1.0 mm.

In the example shown in FIG. 1, the fluid inlet 103 is coupled to a feed vessel 150 via an inlet fluid control means 109. In the example shown the inlet fluid control means 109 is a continuous pump such as a rotary or peristaltic pump. If a peristaltic pump (or any other pulsatile pump) is used it may be coupled to a means to minimise pulsing, for example a flexible tube coupled to the peristaltic pump that is configured to elastically absorb the pulses in pressure in a manner similar to the “windkessel” effect). The overflow outlet 105 is coupled to a waste vessel 125 via an overflow outlet fluid control means 111 and a waste line 127. The overflow outlet fluid control means 111 is also a continuous pump such as a peristaltic pump. The underflow outlet 107 is also coupled to the feed vessel 150 via feed line 152. In the example shown the feed vessel 150 also comprises an input line 170.

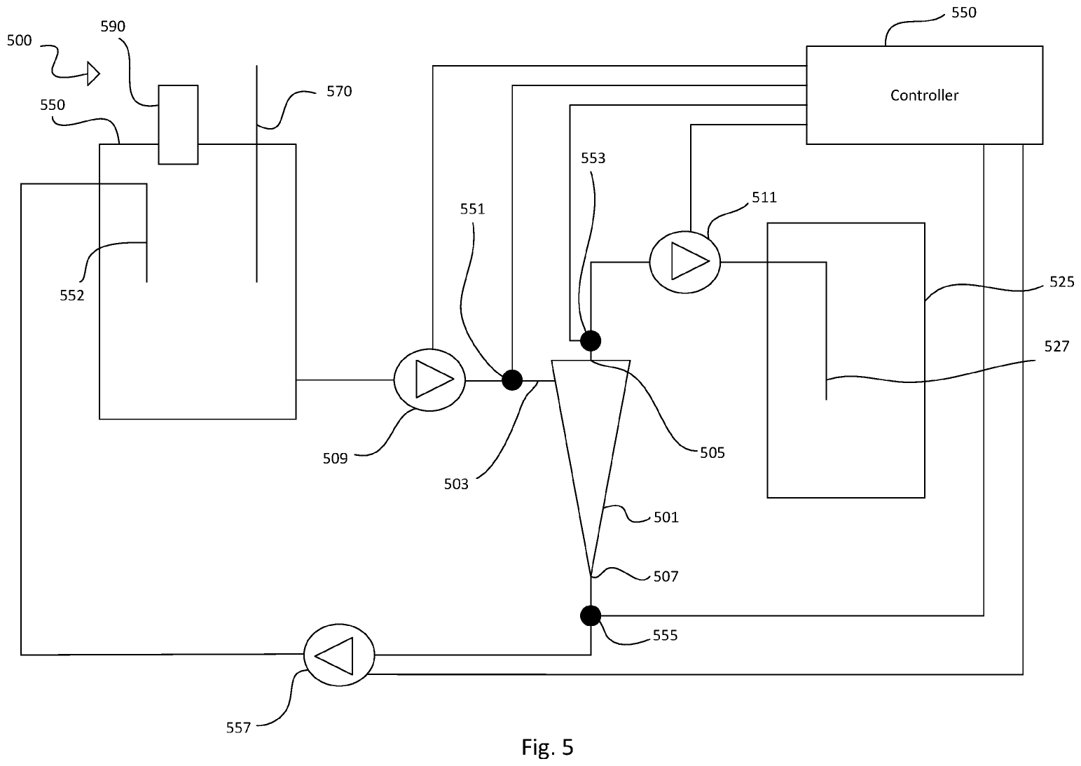

In some examples the system 100 also comprises a controller (not shown, but an example of which is described below with reference to FIG. 5) for controlling the system 100, and in particular for controlling the inlet fluid control means 109 and the overflow outlet fluid control means 111.

In the example shown in FIG. 1, which may be used for example for perfusion of biological cells in cell suspensions, the waste vessel 125 can be used for removing fluid (such as cell media) or particles of lower mass, separated by a cyclone formed in the separation vessel 101. Any higher mass particles (such as cells) would separate out via the underflow outlet 107 and be recycled back into the feed vessel 150, whereas the fluid (such as the cell media) would separate out via the overflow outlet 105 and into the waste vessel 125. The input line 170 may be used to replenish any fluid (such as cell media) removed to the waste vessel 125.

The inlet fluid control means 109 is operable to control the flow of fluid though the fluid inlet 103. The overflow outlet fluid control means 111 is operable to control the flow of fluid through the overflow outlet 105. Controlling the flow of fluid through the fluid inlet 103 and the overflow outlet 105 may thus control the formation and functioning of a cyclone in the separation vessel 101.

In use, a fluid (for example a suspension containing cells) is fed into the separation vessel 101 from the feed vessel 150 via the fluid inlet 103 transverse to and eccentric to a longitudinal axis of the separation vessel 101 such that it creates a cyclone effect in the vessel 101. The inlet fluid control means 109 is operated to control the flow rate and/or pressure of fluid fed into the separation vessel 101. The flow of fluid (such as the flow rate and/or pressure) through the overflow outlet 105 is also controlled by controlling the overflow outlet fluid control means 111. Controlling the inlet fluid control means 109 and/or the overflow outlet fluid control means 111 can therefore control the formation of a cyclone and/or a cyclone in the separation vessel 101.

Preferably the flow of fluid through the fluid inlet 103 and the overflow outlet 105 is controlled by controlling the inlet fluid control means 109 and/or the overflow outlet flow control means 111 such that the flow rate of fluid through the underflow outlet 107 is greater than the flow rate of fluid through the overflow outlet 105.

Once a cyclone is established in the separation vessel 101, in the example of the system 100 being used for cell perfusion, cells may separate out from the separation vessel 101 via the underflow outlet 107 and be fed back (i.e. recycled) into feed vessel 150 via input line 152. Waste media may separate out from the separation vessel 101 and be extracted by the overflow outlet fluid control means 111 via the overflow outlet 105 and into waste vessel 125.

The degree to which fluid is separated out into the waste vessel 125 may be determined based on at least one of (i) a parameter of the fluid and (ii) time. For example, control of the overflow outlet fluid control means 111 may be based on a parameter of the fluid entering and/or in and/or leaving the separation vessel 101. Similarly, control of the inlet fluid control means 109 may be based on a parameter of at least one of (i) the fluid entering (ii) the fluid in and (iii) the fluid leaving the separation vessel 101.

For example, if the fluid reaches a selected threshold density (for example, as determined by turbidity), it may be determined that a selected degree of fluid should be extracted via the overflow outlet 105. Additionally or alternatively, the extraction of fluid via the overflow outlet 105 may be a continuous process, and the flow rate of fluid extracted via the overflow outlet 105 may be based on a parameter, such as the density, of fluid entering and/or in and/or leaving the separation vessel 101. In other examples, the extraction of fluid via the overflow outlet 105 may be based on at least one of: (i) levels of toxic by-products (such as lactate or ammonia) from cell metabolism reaching a selected threshold; (ii) cell phenotype changes (for example during differentiation of pluripotent cells); (iii) the size and/or mass of particles such as cells that are desired to be separated from the suspension, for examples particles with a size and/or mass above or below a selected threshold.

The parameter of the fluid may be determined based on fluid entering the fluid inlet 103, fluid passing through the underflow outlet 107 and/or fluid passing through the overflow outlet 105.

Additionally or alternatively, if a threshold time interval has passed it may be determined to extract a selected amount of fluid via the overflow outlet 105, for example where the volume of fluid extracted is determined based on a function of the time interval.

In the example shown in FIG. 1 the pressure of the fluid passing through the fluid inlet 103 may be maintained between 0.5 and 4 bar.

It will be understood that although a controller is not shown in FIG. 1, the functionality described above may be performed by a controller operable to control the inlet fluid control means 109 and the overflow outlet fluid control means 111, as described below with reference to, and as shown in, FIG. 5. It will also be understood that the system 100 may comprise sensors coupled to, for example, the fluid inlet 103, the overflow outlet 105 and/or the underflow outlet 107, for sensing the parameter of the fluid discussed above, also as described below with reference to, and as shown in, FIG. 5. In some examples there may also be a sensor inside the separation vessel 101, in the feed vessel 150 and/or waste vessel 125.

It will also be understood that in some examples the system 100 may also comprise an optional underflow outlet fluid control means, also as described below with reference to, and as shown in, FIG. 5.

In addition, while in the example shown the overflow outlet 105 is coupled to the waste vessel 125, in some examples it will be understood that overflow outlet 105 may be coupled to the feed vessel 150, for example when the system is intended to be used to remove particles such as cells from the suspension (e.g. when the desired product is in the media such as viruses and exosomes).

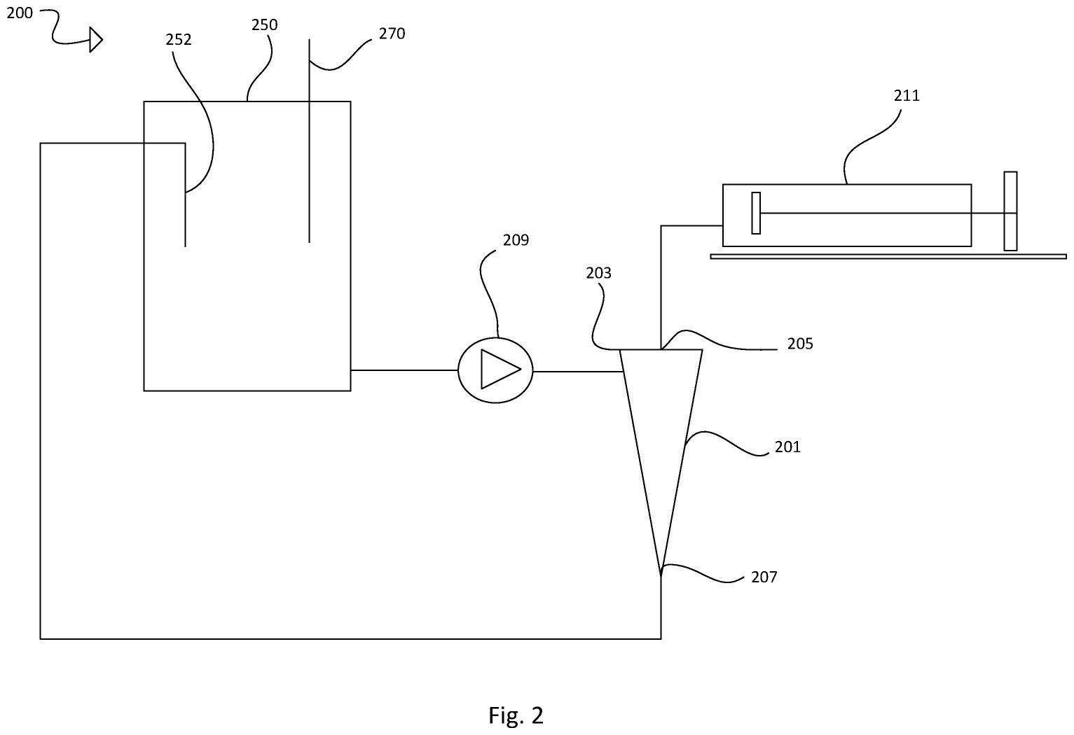

FIG. 2 shows another separating system 200 of embodiments of the disclosure. The system 200 of FIG. 2 is in many respects similar to the system of FIG. 1 described above with like reference numbers indicating similar or the same entities, however instead of the overflow outlet fluid control means 111 being a continuous pump such as a peristaltic pump, in the example shown in FIG. 2 the overflow outlet fluid control means 211 is non-continuous pump, and in the example shown is a syringe pump. As a result, the example shown in FIG. 2 also does not need a waste vessel.

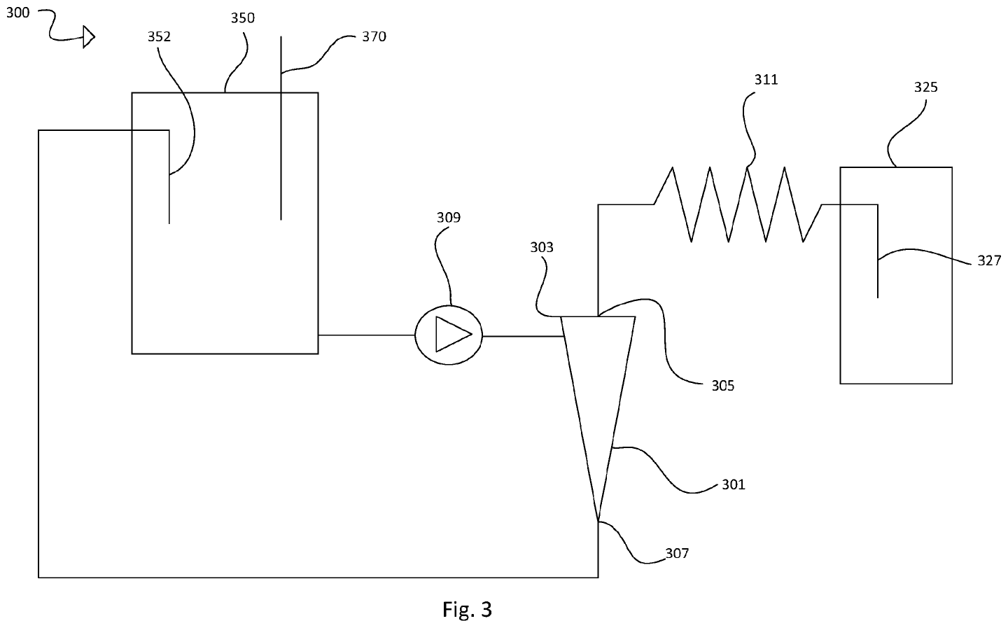

FIG. 3 shows another separating system 300 of embodiments of the disclosure. The system 300 of FIG. 3 is in many respects similar to the system of FIGS. 1 and 2 described above with like reference numbers indicating similar or the same entities, however instead of the overflow outlet fluid control means 111 being a continuous pump such as a peristaltic pump, or a non-continuous pump, in the example shown in FIG. 3 the overflow outlet fluid control means 311 is a fluid resistor. The resistance of the fluid resistor can be adjusted to control the flow of fluid through the overflow outlet fluid control means 311. This adjustment may, for example, be a passive action (for example the tube of the fluid resistor through which the fluid flows may be configured to expand with pressure), and/or an active action (for example by operation of a proportional solenoid valve).

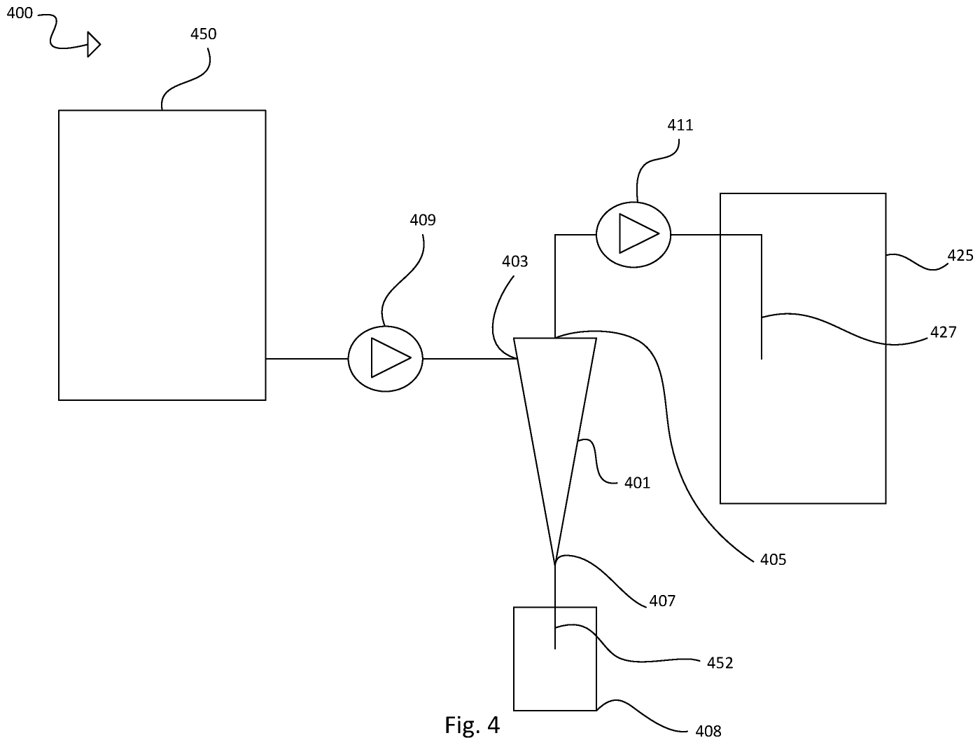

FIG. 4 shows another example separating system 400 of embodiments of the disclosure, and is similar to the system described above with reference to FIG. 1 with like reference numbers indicating similar or the same entities. However, the system of FIG. 4 is slightly different due to its different intended function—whereas the system of FIGS. 1 to 3 may be used, for example, for cell perfusion, the system of FIG. 4 may be configured for use, for example, for cell harvest.

The system 400 comprises a separation vessel 401 having a fluid inlet 403, an underflow outlet 407 and an overflow outlet 405. The separation vessel 401 is conical in shape to enable the formation of a cyclone therewithin. The fluid inlet 403 is proximate to the overflow outlet 405 and configured to direct fluid into the vessel transverse to (for example, perpendicular to) and eccentric to the longitudinal axis of the conical separation vessel 401. The underflow outlet 407 and overflow outlet 105 are coaxial with the longitudinal axis of the conical separation vessel 401.

In the example shown in FIG. 4, the fluid inlet 403 is coupled to a feed vessel 450 via an inlet fluid control means 409. In the example shown the inlet fluid control means 409 is a continuous pump such as a peristaltic pump. The overflow outlet 405 is coupled to a waste vessel 425 via an overflow outlet fluid control means 411 and a waste line 427. The overflow outlet fluid control means 411 is also a continuous pump such as a peristaltic pump. The underflow outlet 407 is coupled to a harvest vessel 408 via a harvest line 452.

In some examples the system 400 also comprises a controller (not shown, but an example of which is described below with reference to FIG. 5) for controlling the system 400, and in particular for controlling the inlet fluid control means 409 and the overflow outlet fluid control means 411.

In the example shown in FIG. 4, which as noted above, may be used for example for cell harvest, the waste vessel 425 can be used for removing less dense fluid (such as cell media) separated by a cyclone formed in the separation vessel 401. Any higher density particles (such as cells) would separate out via the underflow outlet 407 into harvest vessel 408.

The inlet fluid control means 409 is operable to control the flow of fluid though the fluid inlet 403. The overflow outlet fluid control means 411 is operable to control the flow of fluid through the overflow outlet 105. Controlling the flow of fluid through the fluid inlet 403 and the overflow outlet 405 may thus control the formation and functioning of a cyclone in the separation vessel 401.

In use a fluid (for example a biological suspension containing cells) is fed into the separation vessel 401 transverse to and eccentric to the longitudinal axis of the separation vessel 401 from the feed vessel 450 via the fluid inlet 403. The inlet fluid control means 409 is controlled to control the flow rate and/or pressure of fluid fed into the separation vessel 401. The flow of fluid (such as the flow rate and/or pressure) through the overflow outlet 405 is also controlled by controlling the overflow outlet fluid control means 411. Controlling the inlet fluid control means 409 and/or the overflow outlet fluid control means 411 can therefore control the formation of the cyclone in the separation vessel 401.

Preferably the flow of fluid through the fluid inlet 403 and the overflow outlet 405 is controlled by controlling the inlet fluid control means 409 and/or the overflow outlet flow control means 411 such that the flow rate of fluid through the underflow outlet 407 is greater than the flow rate of fluid through the overflow outlet 405.

Once a cyclone is established in the separation vessel 401, in the example of the system 400 being used for cell harvest, cells may separate out from the separation vessel 401 via the underflow outlet 407 and into harvest vessel 408. Cell media may separate out from the separation vessel 401 and be extracted by the overflow outlet fluid control means 411 via the overflow outlet 405 and into waste vessel 425.

The degree to which fluid is separated out into the waste vessel 425 may be determined based on a parameter of the fluid and/or time. For example, control of the overflow outlet fluid control means 411 may be based on a parameter of the fluid entering and/or in and/or leaving the separation vessel 401. Similarly, control of the inlet fluid control means 409 may be based on a parameter of the fluid entering and/or in and/or leaving the separation vessel 401. The parameter of the fluid may be determined based on fluid entering the fluid inlet 403, fluid passing through the underflow outlet 407 and/or fluid passing through the overflow outlet 405.

It will be understood that although a controller is not shown in FIG. 4, the functionality described above may be performed by a controller operable to control the inlet fluid control means 409 and the overflow outlet fluid control means 411. It will also be understood that the system 100 may comprise sensors coupled to, for example, the fluid inlet 403, the overflow outlet 405 and/or the underflow outlet 407, for sensing the parameter of the fluid discussed above. In some examples there may also be a sensor inside the separation vessel 401, in the feed vessel 450, in the harvest vessel 408 and/or waste vessel 425.

FIG. 5 shows another example separating system 500 of embodiments of the disclosure, and is similar to the system described above with reference to FIGS. 1 to 4 with like reference numbers indicating similar or the same entities. However, the system of FIG. 5 also has an optional controller 550 for controlling operation of the system 500.

As with the system of FIG. 1, the system 500 shown in FIG. 5 comprises a separation vessel 501 having a fluid inlet 503, an underflow outlet 507 and an overflow outlet 505. The separation vessel 501 is conical in shape to enable the formation of a cyclone therewithin. The fluid inlet 503 is proximate to the overflow outlet 505 and configured to direct fluid into the separation vessel 501 transverse to and eccentric to the longitudinal axis of the conical vessel 501. The underflow outlet 507 and overflow outlet 505 are coaxial with the longitudinal axis of the conical separation vessel 501.

In the example shown in FIG. 1, the fluid inlet 503 is coupled to a feed vessel 550 via an inlet fluid control means 509. In the example shown the inlet fluid control means 509 is a continuous pump such as a peristaltic pump. The overflow outlet 505 is coupled to a waste vessel 525 via an overflow outlet fluid control means 511 and a waste line 527. The overflow outlet fluid control means 511 is also a continuous pump such as a peristaltic pump. The underflow outlet 507 is also coupled to the feed vessel 550 via an underflow outlet fluid control means 557 and a feed line 552. In the example shown the feed vessel 550 also comprises an input line 570 and a valve 590.

Sensors are also coupled to the input and outputs of the separation vessel 501. An inlet sensor 551 is coupled to the fluid inlet 503, an overflow sensor 553 is coupled to the overflow outlet 503 and an underflow sensor 555 is coupled to the underflow outlet 507. The sensors 551, 553, 555 may be selected from at least one of: a turbidity sensor, a temperature sensor, a pressure sensor, a capacitive sensor and an impedance sensor.

The system 500 also comprises a controller 550 for controlling the system 500. The controller 550 is coupled to the inlet fluid control means 509, the overflow outlet fluid control means 511 and the underflow outlet fluid control means 557. The controller 550 is also coupled to the inlet sensor 551, the overflow sensor 553 and the underflow sensor 555. The controller is also coupled to valve 590.

In the example shown in FIG. 5, which may be used for example for perfusion of biological cells in cell suspensions, the waste vessel 525 can be used for removing less dense fluid (such as cell media) separated by a cyclone formed in the separation vessel 501. Any higher density particles (such as cells) would separate out via the underflow outlet 507 and be recycled back into the feed vessel 550, whereas the less dense fluid (such as the cell media) would separate out via the overflow outlet 505 and into the waste vessel 525. The input line 570 may be used to replenish any fluid (such as cell media) removed to the waste vessel 525.

The inlet fluid control means 509 is operable to control the flow of fluid though the fluid inlet 103. The overflow outlet fluid control means 511 is operable to control the flow of fluid through the overflow outlet 105. The underflow outlet fluid control means 557 is operable to control the flow of fluid through the underflow outlet 557. The valve 590 may be controlled to control the pressure in the feed vessel 550, and thus the pressure of fluid flowing into the separation vessel 501.

The inlet sensor 551 is operable to sense a parameter of the fluid flowing through the fluid inlet 503. The overflow sensor 553 is operable to sense a parameter of fluid flowing through the overflow outlet 505. The underflow sensor 555 is operable to sense a parameter of fluid flowing through the underflow outlet 507.

The controller 550 is operable to control the inlet fluid control means 509, the overflow outlet fluid control means 511, the underflow outlet fluid control means 557 and optionally valve 590 to control the flow of fluid into and out of the separation vessel 501. The valve 590 may be operable to control the pressure in the feed vessel 590 and therefore the pressure of fluid flowing into the separation vessel 501, for example by introducing a fluid such as a gas into the feed vessel 590, or allowing a pressurised fluid such as a gas to escape the feed vessel 590. It will be understood that in some examples the harvest vessel and/or waste vessel (if present) may also comprise a similar valve.

The controller 550 is also operable to control to the inlet sensor 551, the overflow sensor 553 and the underflow sensor 555. The inlet sensor 551, the overflow sensor 553 and the underflow sensor 555 are configured to send sensor signals indicative of a parameter of the fluid to the controller 550. The controller 550 is configured to make a determination of a parameter of the fluid based on the received sensor signals.

In use a fluid (for example a biological suspension containing cells) is fed into the separation vessel 501 transverse to and eccentric to the longitudinal axis of the separation vessel 501 from the feed vessel 550 via the fluid inlet 503. The inlet fluid control means 509 is controlled by the controller 550 to control the flow rate and/or pressure of fluid fed into the separation vessel 501. The flow of fluid (such as the flow rate and/or pressure) through the overflow outlet 505 is also controlled by the controller 550 by controlling the overflow outlet fluid control means 511 and/or the underflow outlet fluid control means 557. Controlling the inlet fluid control means 509 and/or the overflow outlet fluid control means 511 and/or underflow outlet fluid control means 557 can therefore control the formation of the cyclone in the separation vessel 501.

Preferably the flow of fluid through the fluid inlet 503 and the overflow outlet 505 is controlled by controlling the inlet fluid control means 509 and/or the overflow outlet flow control means 511 such that the flow rate of fluid through the underflow outlet 507 is greater than the flow rate of fluid through the overflow outlet 505.

Once a cyclone is established in the separation vessel 501, in the example of the system 500 being used for cell perfusion, cells may separate out from the separation vessel 501 via the underflow outlet 507 and be fed back (i.e. recycled) into feed vessel 550 via input line 552. Waste media may separate out from the separation vessel 501 and be extracted by the overflow outlet fluid control means 511 via the overflow outlet 505 and into waste vessel 525.

In the example shown in FIG. 5, control of the fluid control means, such as the inlet fluid control means 509, the overflow outlet fluid control means 511 and/or the underflow outlet fluid control means 557 is based on a parameter of the fluid entering and/or in and/or leaving the separation vessel 501. Control of the valve 590 may also be based on a parameter of the fluid entering and/or in and/or leaving the separation vessel 501. This is done by the controller 550 controlling operation of the inlet sensor 551, the overflow sensor 553 and the underflow sensor 555 to receive sensor signals indicative of a parameter of the fluid at those points. The controller 550 makes a determination of a parameter of the fluid based on the received sensor signals, and determines what control of the inlet fluid control means 509, the overflow outlet fluid control means 511, the underflow outlet fluid control means 557 and/or valve 590 is needed based on the determined parameters of the fluid.

For example, if the fluid entering the separation vessel 501 reaches a selected threshold density (for example, as determined by inlet sensor 551 which may be a turbidity sensor), it may be determined by the controller that fluid should be extracted via the overflow outlet 505. The amount of fluid that is extracted may be based on the determined density of the fluid, for example the amount of fluid extracted may be proportional to the difference between the measured density and the threshold density. This may be done by controlling operation of the overflow outlet control means 511 and/or the underflow outlet control means 557.

Additionally or alternatively, the extraction of fluid via the overflow outlet 505 may be a continuous process, and the flow rate of fluid extracted via the overflow outlet 505 may be based on a parameter, such as the density, of fluid entering and/or in and/or leaving the separation vessel 501. For example, the controller may have a feedback control loop that continuously monitors a parameter of the fluid (such as the density) and controls operation of the overflow outlet control means 511 as a continuous process based on the feedback control loop.

Additionally or alternatively, if a threshold time interval has passed it may be determined to extract a selected amount of fluid via the overflow outlet 505, for example where the volume of fluid extracted is determined based on a function of the time interval. For example, the controller 550 may extract a selected amount of fluid repeatedly at a selected time interval via the overflow outlet 505 by controlling the overflow outlet fluid control means 511.

It will be understood that although the system 500 shown in FIG. 5 comprises an inlet fluid control means 509, an overflow outlet fluid control means 511 and an underflow outlet fluid control means 557, it will be understood that in some examples the system 500 may not comprise all three control means. In addition, although the system 500 shown in FIG. 5 comprises an inlet sensor 551, an overflow sensor 553 and an underflow sensor 555, in some examples the system 500 may only comprise two or even only one sensor. In addition, it will be understood that the valve 590 is optional.

The controller 550 may be configured to operate in two modes:
(i) an initialisation mode for establishing a cyclone in the separation vessel; and
(ii) a cyclone mode for separating material from suspensions, e.g. cells from a suspension.

In the initialisation mode the controller 550 may be configured to inhibit the flow of fluid through the overflow outlet 505, but only, for example, through the fluid inlet 503 and/or the underflow outlet 507. In some examples, in the initialisation mode the controller 550 may be configured to control the flow of fluid through the overflow outlet 505 such that no fluid flows through the overflow outlet 505 (for example, so that it is blocked).

In the cyclone mode the controller 550 may be configured to adjustably control the flow of fluid through at least one of (i) the overflow outlet 505 and (ii) the underflow outlet 507. The controller 550 may be configured to determine when to switch between the initialisation mode and the cyclone mode based on a parameter, such as the speed and/or pressure, of the fluid passing through at least one of (i) the underflow outlet 507 and (ii) the overflow outlet 505. Preferably the controller 550 is configured to switch between the initialisation mode and the cyclone mode based on a parameter of the fluid passing through the overflow outlet 505. When the controller 550 switches between the initialisation mode and the cyclone mode, the controller 550 may be configured to gradually increase or ramp up the flow rate of the flow of fluid through the overflow outlet 505 to a selected rate, for example from a flow rate of zero (i.e. blocked) to the selected flow rate. This may be desirable so as not to create any sudden/destabilising perturbations to the system which may result in the cyclone collapsing.

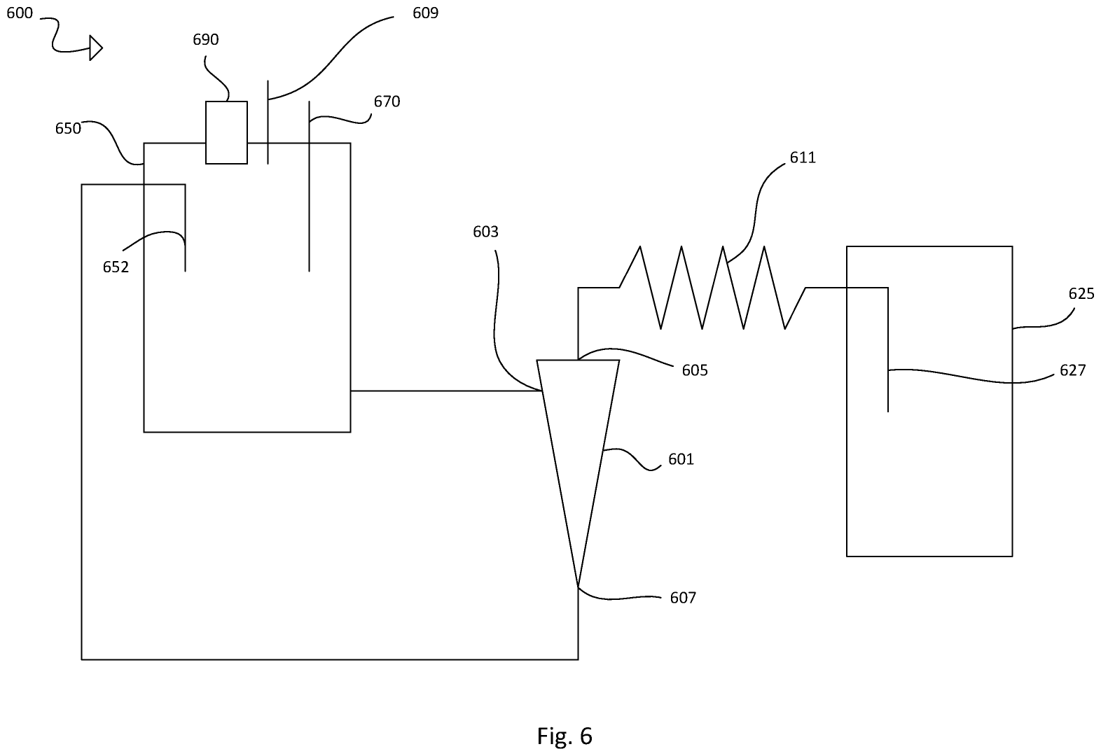

FIG. 6 shows another example separating system 600 of embodiments of the disclosure, and is similar to the system described above with reference to FIG. 1 with like reference numbers indicating similar or the same entities. FIG. 6 shows an example separating system 600 of embodiments of the disclosure. The system 600 comprises a separation vessel 601 having a fluid inlet 603, an underflow outlet 607 and an overflow outlet 605. The separation vessel 601 is conical in shape to enable the formation of a cyclone therewithin. The fluid inlet 603 is proximate to the overflow outlet 605 and configured to direct fluid into the separation vessel transverse to and eccentric to the longitudinal axis of the conical separation vessel 601. The underflow outlet 607 and overflow outlet 605 are coaxial with the longitudinal axis of the conical separation vessel 601.

In the example shown in FIG. 6, the fluid inlet 603 is coupled to a feed vessel 650. The overflow outlet 605 is coupled to a waste vessel 625 via an overflow outlet fluid control means 611 and a waste line 627. The overflow outlet fluid control means 611 is a fluid resistor. The underflow outlet 607 is also coupled to the feed vessel 650 via feed line 652. In the example shown the feed vessel 650 also comprises an input line 670 and optional valve 690. The feed vessel 650 also comprises compressed gas feed 609, which the skilled person may consider to be a fluid inlet control means as it is operable to control the flow of fluid through the fluid inlet 603.

In some examples the system 600 also comprises a controller (not shown, but an example of which is described above with reference to FIG. 5) for controlling the system 600, and in particular for controlling the compressed gas feed 609 and the overflow outlet fluid control means 611.

In the example shown in FIG. 6, which may be used for example for perfusion of biological cells in cell suspensions, the waste vessel 625 can be used for removing less dense fluid (such as cell media) separated by a cyclone formed in the separation vessel 601. Any higher density particles (such as cells) would separate out via the underflow outlet 607 and be recycled back into the feed vessel 650, whereas the less dense fluid (such as the cell media) would separate out via the overflow outlet 605 and into the waste vessel 625. The input line 670 may be used to replenish any fluid (such as cell media) removed to the waste vessel 625.

The compressed gas feed 609 is operable to control the flow of fluid into the separation vessel 601 though the fluid inlet 603. The overflow outlet fluid control means 611 is operable to control the flow of fluid through the overflow outlet 605. Controlling the flow of fluid through the fluid inlet 603 and the overflow outlet 605 may thus control the formation and functioning of a cyclone in the separation vessel 601.

In use, a fluid (for example a cell suspension containing viable cells) is fed into the separation vessel 601 transverse to and eccentric to the longitudinal axis of the separation vessel 601 from the feed vessel 650 via the fluid inlet 603. The compressed gas feed 609 is controlled to control the pressure (and thereby flow rate) of fluid fed into the separation vessel 601. The flow of fluid (such as the flow rate and/or pressure) through the overflow outlet 605 is also controlled by operating the overflow outlet fluid control means 611. Controlling the compressed gas feed 609 and/or the overflow outlet fluid control means 611 can therefore control the formation of the cyclone in the separation vessel 601.

Preferably the flow of fluid through the fluid inlet 603 and the overflow outlet 605 is controlled by controlling the compressed gas feed 609 and/or the overflow outlet flow control means 611 such that the flow rate of fluid through the underflow outlet 607 is greater than the flow rate of fluid through the overflow outlet 605.

Once a cyclone is established in the separation vessel 601, in the example of the system 600 being used for cell perfusion, cells may separate out from the separation vessel 601 via the underflow outlet 607 and be fed back (i.e. recycled) into feed vessel 650 via input line 652. Waste media may separate out from the separation vessel 601 and be extracted by the overflow outlet fluid control means 611 via the overflow outlet 605 and into waste vessel 625.

As with the other examples described above, the degree to which fluid is separated out into the waste vessel 625 may be determined based on a parameter of the fluid and/or time. For example, operation of the overflow outlet fluid control means 611 may be based on a parameter of the fluid entering and/or in and/or leaving the separation vessel 601. Similarly, control of the inlet fluid control means 609 may be based on a parameter of the fluid entering and/or in and/or leaving the separation vessel 601. However, it will also be understood that the degree to which fluid is separated out from the separation vessel 601 may be based on other factors, for example at least one of: (i) levels of toxic by-products (such as lactate or ammonia) from cell metabolism reaching a selected threshold; and (ii) cell phenotype changes (for example during differentiation of pluripotent cells).

For example, if the fluid reaches a selected threshold density, such as density of cells within the fluid, (for example, as determined by turbidity), it may be determined that a selected degree of fluid should be extracted via the overflow outlet 605. Additionally or alternatively, the extraction of fluid via the overflow outlet 605 may be a continuous process, and the flow rate of fluid extracted via the overflow outlet 605 may be based on a parameter, such as the density, of fluid entering and/or in and/or leaving the separation vessel 601.

The parameter of the fluid may be determined based on fluid entering the fluid inlet 603, fluid passing through the underflow outlet 107 and/or fluid passing through the overflow outlet 605.

Additionally or alternatively, if a threshold time interval has passed it may be determined to extract a selected amount of fluid via the overflow outlet 605, for example where the volume of fluid extracted is determined based on a function of the time interval.

It will be understood that although a controller is not shown in FIG. 6, the functionality described above may be performed by a controller operable to control the compressed gas feed 609 and the overflow outlet fluid control means 611 (and also optionally valve 690). It will also be understood that the system 600 may comprise sensors coupled to, for example, the fluid inlet 603, the overflow outlet 605 and/or the underflow outlet 607, as described above with reference to FIG. 5, for sensing the parameter of the fluid discussed above. In some examples there may also be a sensor inside the separation vessel 601, in the feed vessel 650 and/or waste vessel 625.

It will also be understood that in some examples the system 600 may also comprise an optional underflow outlet fluid control means, as described above with reference to, and as shown in, FIG. 5. It will also be understood that in some examples the system 600 may only comprise one fluid control means.

The examples described above and as shown in FIGS. 1 to 6 only have one fluid inlet, however, it will be understood that in other examples the separation vessel may have a plurality of fluid inlets. Each of the plurality of fluid inlets may have a respective fluid inlet control means. Having a plurality of fluid inlets each with a respective fluid inlet control means may allow the formation of a cyclone in the separation vessel to be more easily controlled. For example, the separation vessel may have a first fluid inlet configured to feed fluid in a first direction transverse to, and eccentric to the longitudinal axis of the separation vessel on one side of the separation vessel, and a second fluid inlet configured to feed fluid in a second direction opposite to the first direction and transverse to, and eccentric to the longitudinal axis of the separation vessel on an opposing side of the separation vessel. This may be beneficial as it may reduce the asymmetry of energy input at the top of the cyclone formed in the separation vessel, which may increase the speed at which a cyclone forms in the separation vessel, and the stability of a cyclone formed in the separation vessel.

As noted above, it will also be understood that the separation vessel may comprise a plurality of underflow and/or overflow outlets (optionally with respective fluid control means) and each coupled to a tube or line with a corresponding bore or diameter matching that of the respective outlet. In some examples the plurality of underflow and/or overflow outlets may have differing diameters and may be concentric with each other—for example, if there are two overflow outlets, one with a larger bore or diameter than the other, the two overflow outlets may be concentric with each other (for example such that one sits inside the other).

Although all of the examples shown above with reference to FIGS. 1 to 6 show some form of inlet fluid control means and an outlet fluid control means, it will be understood that in some examples an inlet fluid control means is not essential. For example, a fluid suspension may be into the separation vessel under gravity if the feed vessel is positioned above the separation vessel.

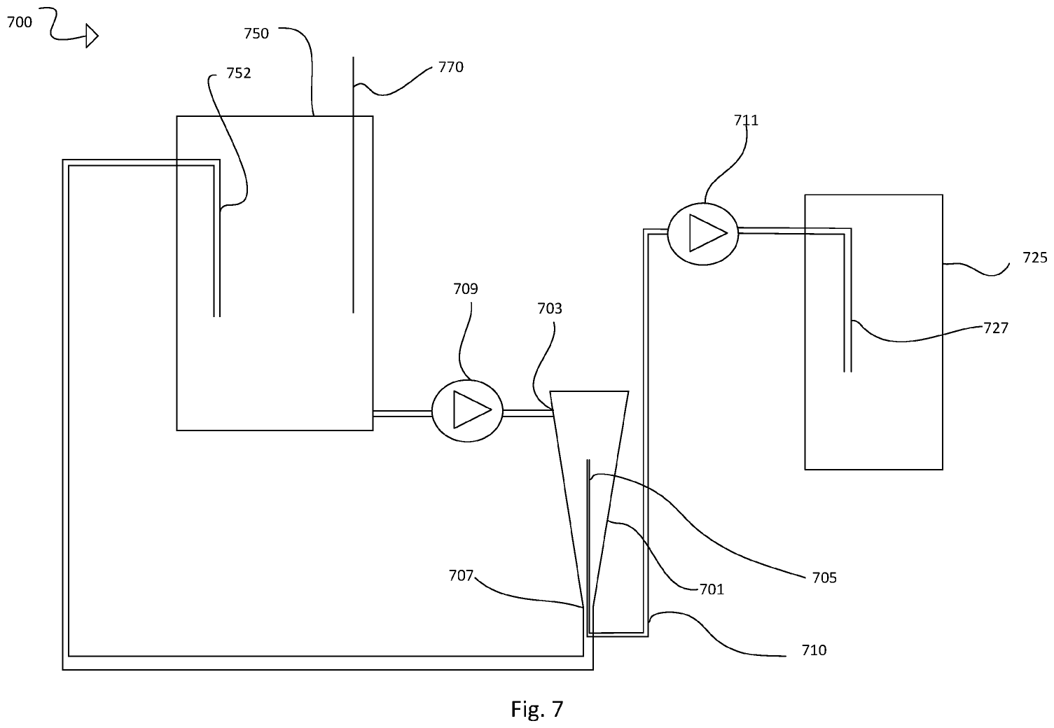

FIG. 7 shows another example separating system 700 of embodiments of the disclosure, and is similar to the system described above with reference to FIG. 1 with like reference numbers indicating similar or the same entities. FIG. 7 shows an example separating system 700 of embodiments of the disclosure. The system 700 comprises a separation vessel 701 having a fluid inlet 703, an underflow outlet 707 and an overflow outlet 705. The separation vessel 701 is conical in shape to enable the formation of a cyclone therewithin. The separation vessel 701 therefore has a longitudinal axis about an axis of symmetry of the cone. It will also be appreciated that in some examples the entire shape of the separation vessel 701 need not be conical, for example a portion of the separation vessel may be conical and another portion (such as the portion into which the fluid inlet 703 is couple) may be cylindrical.

The underflow outlet 707 is located at the bottom or apex of the conical shape of the separation vessel, at a proximal end of the longitudinal axis of the separation vessel 701 (although it will be understood that in other examples the underflow outlet 707 does not need to be at an end of the separation vessel 701, for example the underflow outlet 707 may be inset distally from the end of the separation vessel 701). The fluid inlet 703 is near the top of the conical shape of the separation vessel 701 towards a distal end of the longitudinal axis of the separation vessel 701.

In the example shown in FIG. 7, the overflow outlet 705 is coupled to a tube 710 that extends through the bottom of, or proximal end of, the separation vessel 701 coaxial with the underflow outlet 707 and up inside the separation vessel 701 parallel with and coaxial with the longitudinal axis of the separation vessel 701 (although it will be understood that in other examples the tube 710 coupled to the overflow outlet 705 need not extend coaxial with the longitudinal axis of the separation vessel 701 but may be eccentric to or offset from the longitudinal axis of the separation vessel 701 such that it is configured to extract fluid from a different radial location relative to the longitudinal axis compared to the radial location at which the underflow outlet 707 is configured to extract fluid/material). The tube 710 supports the overflow outlet 705 such that it is located between the fluid inlet 703 and the underflow outlet 707 along the longitudinal axis of the separation vessel 701, yet nearer the fluid inlet 703 along the longitudinal axis than to the underflow outlet 707. The underflow outlet 707 and overflow outlet 705 are therefore coaxial with the longitudinal axis of the conical separation vessel 701.

In the example shown in FIG. 7, the fluid inlet 703 is coupled to a feed vessel 750. The overflow outlet 705 is coupled to a waste vessel 725 via the tube 710 coupled to an overflow outlet fluid control means 711 and a waste line 727. The overflow outlet fluid control means 711 is a pump. The underflow outlet 707 is also coupled to the feed vessel 750 via feed line 752. In the example shown the feed vessel 750 also comprises an input line 770.

In some examples the system 700 also comprises a controller (not shown, but an example of which is described above with reference to FIG. 5) for controlling the system 700, for example for controlling the overflow outlet fluid control means 711.

The fluid inlet 703 is configured to direct fluid into the separation vessel 701 transverse to and eccentric to the longitudinal axis of the conical separation vessel 701. The overflow outlet 705 is positioned within the separation vessel 701 so as to draw fluid from a region, in use, proximal to the top of a cyclone formed in the separation vessel 701 (wherein the top of the cyclone may be defined as the portion of the cyclone with the greatest diameter).

The example system shown in FIG. 7, similar to the example shown in FIG. 1, may be used for example for perfusion of biological cells in cell suspensions. In this example, the waste vessel 725 can be used for removing fluid (such as cell media) or particles of lower mass, separated by a cyclone formed in the separation vessel 701. Any higher mass particles (such as cells) would separate out via the underfloor outlet 707 and be recycled back into the feed vessel 750, whereas the fluid (such as the cell media) would separate out via the overflow outlet 705 and into the waste vessel 725. The input line 770 may be used to replenish any fluid (such as cell media) removed to the waste vessel 725.

The inlet fluid control means 709 is operable to control the flow of fluid though the fluid inlet 703. The overflow outlet fluid control means 711 is operable to control the flow of fluid through the overflow outlet 105. Controlling the flow of fluid through the fluid inlet 703 and the overflow outlet 705 may thus control the formation and functioning of a cyclone in the separation vessel 701.

In use, a fluid (for example a cell suspension containing viable cells) is fed into the separation vessel 701 transverse to and eccentric to the longitudinal axis of the separation vessel 701 from the feed vessel 750 via the fluid inlet 703. The flow of fluid (such as the flow rate and/or pressure) through the overflow outlet 705 is also controlled by operating the overflow outlet fluid control means 711. Controlling the inlet fluid control means 709 and/or the overflow outlet fluid control means 711 can therefore control the formation of the cyclone in the separation vessel 701.

It will be appreciated that in the context of the examples described above the fluid is a liquid comprising biological material suspended in a suspension. However, it will be appreciated that embodiments described herein may be used for removing small or powdered solids from air, water, or other gases or liquids by centrifugal force.

It will be appreciated from the discussion above that the embodiments shown in the Figures are merely exemplary, and include features which may be generalised, removed or replaced as described herein and as set out in the claims. In the context of the present disclosure other examples and variations of the apparatus and methods described herein will be apparent to a person of skill in the art.

1. A cell suspension separating system for separating material from a biological suspension, the system comprising:
a separation vessel arranged to enable the formation of a cyclone therewithin, the separation vessel comprising a fluid inlet, an underflow outlet and an overflow outlet;
an inlet fluid control means for controlling the flow of fluid through the inlet; and
an overflow outlet fluid control means for controlling the flow of fluid through the overflow outlet;
further comprising a sensor coupled to a controller and arranged to sense a parameter of the fluid flowing through at least one of the fluid inlet and the overflow outlet, and wherein the controller is configured to control at least one of the fluid control means based on sensor signals received from the sensor.

2. The system of claim 1 further comprising an underflow outlet fluid control means for controlling the flow of fluid through the underflow outlet.

3. (canceled)

4. (canceled)

5. (canceled)

6. The system of claim 1, further comprising:
a first sensor coupled to a controller and arranged to sense a parameter of the fluid flowing through the fluid inlet; and
a second sensor coupled to the controller and arranged to sense a parameter of the fluid flowing through at least one of:
(i) the underflow outlet;
(ii) the overflow outlet; and
(iii) the separation vessel; and
wherein the controller is configured to control the inlet fluid control means and at least one of:
(iv) the underflow outlet fluid control means; and
(v) the overflow outlet control means;
based on sensor signals received from the sensor.

7. The system of claim 1 wherein the sensor is selected from at least one of: a turbidity sensor, a temperature sensor, a pressure sensor, a flowrate sensor, a capacitive sensor and an impedance sensor.

8. The system of claim 1, wherein at least one of the sensors is a turbidity sensor, and the controller is configured to make a determination of the density of the fluid based on sensor signals received from the turbidity sensor, and control at least one of the fluid control means based on the determined density of the fluid.

9. The system of claim 1, further comprising a feed vessel for containing a biological suspension containing material coupled to the fluid inlet, and wherein the controller is configured to control the pressure of the feed vessel for controlling the flow of fluid through the fluid inlet.

10. The system of claim 1, wherein the fluid control means are configured to adjustably vary the flow rate and/or pressure of fluid flowing through the corresponding inlet or outlet.

11. (canceled)

12. A cell suspension separating method for separating material from a biological suspension, the method comprising:
feeding a biological fluid suspension containing material into a separation vessel via a fluid inlet for establishing a cyclone in the separation vessel about a longitudinal axis of the separation vessel, wherein the vessel comprises an underflow outlet and an overflow outlet and wherein the fluid is fed transverse to the longitudinal axis of the separation vessel;
receiving sensor signals indicative of a parameter of the fluid; and
controlling the flow of fluid through at least one of the fluid inlet and the overflow outlet to control the separation of material from the biological suspension based on the received sensor signals.

13. The method of claim 12, further comprising receiving sensor signals indicative of a parameter of the fluid flowing through at least one of:
(i) the fluid inlet;
(ii) the underflow outlet;
(iii) the overflow outlet;
(iv) the separation vessel; and
controlling the flow of fluid through at least one of:
(iv) the underflow outlet; and
(v) the overflow outlet;
to control the separation of material from the biological suspension.

14. The method of claim 12, further comprising controlling the flow of fluid into the separation vessel via the underflow outlet based on the received sensor signals.

15. (canceled)

16. The method of claim 12, further comprising controlling the flow of fluid through at least one of:
(i) the fluid inlet; and
(ii) the overflow outlet;
based on the flow rate and/or pressure of fluid through the fluid inlet to control the separation of material from the biological suspension.

17. The method of claim 12, further comprising controlling the pressure of the biological suspension fed into the separation vessel to control the separation of material from the biological suspension in the separation vessel.

18. A cell suspension separating controller for controlling the separation of material from a biological suspension in a separation vessel having a fluid inlet, an overflow outlet and an underflow outlet, wherein the controller is configured to control the flow rate and/or pressure of fluid through at least one of:
(i) the overflow outlet; and
(ii) the fluid inlet;
based on the flow rate and/or pressure of fluid through at least one of the fluid inlet, the underflow outlet, the overflow outlet, and inside the separation vessel to control the separation of material from the biological suspension.

19. The controller of claim 18, wherein the controller is configured to receive sensor signals indicative of a parameter of the fluid flowing through at least one of the fluid inlet, the underflow outlet, the overflow outlet, and inside the separation vessel; and
wherein the controller is configured to control, based on sensor signals received from the sensor, at least one of:
(i) an inlet fluid control means for controlling the flow of fluid through the inlet; and
(ii) an overflow outlet fluid control means for controlling the flow of fluid through the overflow outlet.

20. The controller of claim 19 wherein the controller is further configured to control an underflow outlet fluid control means for controlling the flow of fluid through the underflow outlet based on the received sensor signals.

21. The controller of claim 18, wherein the controller is configured to determine the density of the fluid and control the flow of fluid through at least one of the fluid control means based on the determined density of the fluid.

22. The controller of claim 18, wherein the controller is configured to operate in two modes:
(i) an initialisation mode for establishing a cyclone in the separation vessel; and
(ii) a cyclone mode for separating material from a biological suspension.

23. The controller of claim 22 wherein:
in the initialisation mode the controller is configured to inhibit the flow of fluid through the overflow outlet; and
in the cyclone mode the controller is configured to adjustably control the flow of fluid through at least one of (i) the overflow outlet and (ii) the underflow outlet.

24. The controller of claim 23 wherein the controller is configured to determine when to switch between the initialisation mode and the cyclone mode based on a pressure of fluid passing through at least one of (i) the underflow outlet and (ii) the overflow outlet.

25. (canceled)
